(19) United States
(12) Patent Application Publication
(10) Pub. No.: US 2024/0200767 A1
(43) Pub. Date: Jun. 20, 2024

(54) CURING LIGHT WITH HEAT DISSIPATION STRUCTURE

(71) Applicant: KOREA PHOTONICS TECHNOLOGY INSTITUTE, Gwangju (KR)

(72) Inventors: Sun Hee AHN, Gwangju (KR); Ki Hyun KIM, Gwangju (KR); In Hee SHIN, Gwangju (KR)

(73) Assignee: KOREA PHOTONICS TECHNOLOGY INSTITUTE, Gwangju (KR)

(21) Appl. No.: 18/555,544

(22) PCT Filed: Dec. 14, 2022
(86) PCT No.: PCT/KR2022/020397

§ 371 Date: Oct. 16, 2023

(30) Foreign Application Priority Data
Jan. 5, 2022 (KR) 10-2022-0001615

(51) Int. Cl. F21V 29/70; A61N 5/06

(52) U.S. Cl. CPC F21V 29/70; A61N 5/0603; A61N 5/062; G02B 6/0008; A61N 2005/0606; A61N 2005/0663; A61N 2005/0666

(57) ABSTRACT

The present invention relates to a curing light with a heat dissipation structure, including: a light source for emitting light for curing a gum inflammation treatment material including a photosensitizer; a control board for controlling the light source; a housing for accommodating the light source and the control board; and heat sinks for dissipating the heat generated from the light source, wherein the heat sinks include: a first heat sink adapted to accommodate the light source and dissipate the heat generated from the light source; and a second heat sink coupled to the first heat sink in such a way as to accommodate the light source, together with the first heat sink, and having a light transmission hole through which the light emitted from the light source is transmitted.

### TECHNICAL FIELD

The present invention relates to a curing light with a heat dissipation structure, more specifically to a curing light that is capable of curing a gum inflammation treatment material, while improving heat dissipation performance thereof.

### BACKGROUND ART

Generally, implants are replacement human tissues when original human tissues are missing, and further, dental implants are artificial tooth roots used to support a restoration for missing teeth. To replace a missing tooth root, in specific, dental implant surgery is carried out to place a fixture made of a material having no rejection reaction to the human body in the alveolar bone and to then fix an artificial tooth to the fixture, so that the function of the tooth can be restored.

A general dental prosthesis or denture causes neighboring teeth and bones to be damaged as time passes, but a dental implant causes no damage on the neighboring teeth and bones and does not have any secondary decay, so that the dental implant is used stably. However, inflammation may occur on the gum around the dental implant.

To apply an anti-inflammation material onto an inflammatory lesion or gum around the implant, the anti-inflammation material is conjugated with collagen gel so that it physically sticks to the inflammatory lesion or gum. Through the efficacy of the anti-inflammation material sticking to the inflammatory lesion or gum, the inflammation can be consistently inhibited.

In this case, a curing light for a local lesion such as gums has a physical limitation because the lesion as a light-cured subject is narrow and small, and accordingly, the curing light has the shape of a generally long narrow rod.

Therefore, the curing light requires a structure capable of performing effective heat dissipation because of its structural limitation.

### DISCLOSURE

### Technical Problem

Accordingly, the present invention has been made in view of the above-mentioned problems occurring in the related art, and it is an object of the present invention to provide a curing light with a heat dissipation structure that is capable of effectively dissipating the heat emitted from a light source through the heat dissipation structure in which heat sinks are coupled to each other to accommodate the light source therein.

It is another object of the present invention to provide a curing light with a heat dissipation structure that is capable of allowing one side heat sink to be exposed to the outside, thereby having advantages such as conveniences of use and easy heat dissipation.

It is yet another object of the present invention to provide a curing light with a heat dissipation structure that is capable of having heat sinks adapted to effectively dissipate the heat generated from a control board to the outside.

### Technical Solution

To accomplish the above-mentioned object, according to the present invention, there is provided a curing light with a heat dissipation structure, including: a light source for emitting light for curing a gum inflammation treatment material including a photosensitizer; a control board for controlling the light source; a housing for accommodating the light source and the control board; and heat sinks for dissipating the heat generated from the light source, wherein the heat sinks include: a first heat sink adapted to accommodate the light source and dissipate the heat generated from the light source; and a second heat sink coupled to the first heat sink in such a way as to accommodate the light source, together with the first heat sink, and having a light transmission hole through which the light emitted from the light source is transmitted.

According to the present invention, desirably, the first heat sink may be located inside the housing and fixedly come into close contact with the control board to dissipate and transfer the heat generated from the control board to the second heat sink.

According to the present invention, desirably, the outer surface of the second heat sink may be exposed to the outside from the housing in which the light source and the control board are accommodated.

According to the present invention, desirably, the second heat sink may have the shape of a truncated cone, and the light transmission hole may have a light reflection surface treatment portion formed on the peripheral surface thereof.

According to the present invention, desirably, the curing light may further include: a head for accommodating an optical fiber adapted to irradiate the light emitted from the light source onto the outside; and a head fixing part for fixing the head thereto and coupled to the second heat sink in such a way as to dissipate the heat received from the second heat sink to the outside.

According to the present invention, desirably, the second heat sink may have a fastening portion formed on one side of the light transmission hole in such a way as to be fastened to a coupling portion formed on the head fixing part.

According to the present invention, desirably, the first heat sink and the second heat sink may be made by means of die casting and post-processing using aluminum.

According to the present invention, desirably, the housing may be configured to have first and second housings coupled to each other so that the light source, the control board, and the first heat sink are accommodated in the housing and the second heat sink is exposed to the outside from one side of the housing.

### Advantageous Effects

According to the present invention, the curing light with the heat dissipation structure can effectively dissipate the heat emitted from the light source and the heat generated from the control board to the outside through the heat dissipation structure in which the heat sinks are coupled to each other to accommodate the light source therein.

Further, the curing light with the heat dissipation structure according to the present invention can allow one side heat sink to be exposed to the outside, thereby having advantages such as conveniences of use and easy heat dissipation.

### BRIEF DESCRIPTION OF DRAWINGS

FIG. 1 is a perspective view showing a curing light with a heat dissipation structure according to the present invention.

FIGS. 2 and 3 are perspective views showing the curing light with a heat dissipation structure according to the present invention.

FIG. 4 is an exploded perspective view showing the curing light with a heat dissipation structure according to the present invention.

FIG. 5 is a perspective view showing a coupled structure of a housing and heat sinks of the curing light with a heat dissipation structure according to the present invention.

FIG. 6 is an exploded perspective view showing a charging cradle of the curing light with a heat dissipation structure according to the present invention.

### MODE FOR INVENTION

The present invention may be modified in various ways and may have several exemplary embodiments. Specific exemplary embodiments of the present invention are illustrated in the drawings and described in detail in the detailed description. However, this does not limit the invention within specific embodiments and it should be understood that the invention covers all the modifications, equivalents, and replacements within the idea and technical scope of the invention.

If it is determined that the detailed explanation on the well-known technology related to the present invention makes the scope of the present invention not clear, the explanation will be avoided for the brevity of the description. In the description, the thicknesses of the lines or the sizes of the components shown in the drawing may be magnified for the clarity and convenience of the description.

When it is said that one element is described as being “connected” or “coupled” to the other element, one element may be directly connected or coupled to the other element, but it should be understood that another element may be present between the two elements. In contrast, when it is said that one element is described as being “directly connected” or “directly coupled” to the other element, it should be understood that another element is not present between the two elements. In the description, when it is said that one portion is described as “includes” any component, one element further may include other components unless no specific description is suggested.

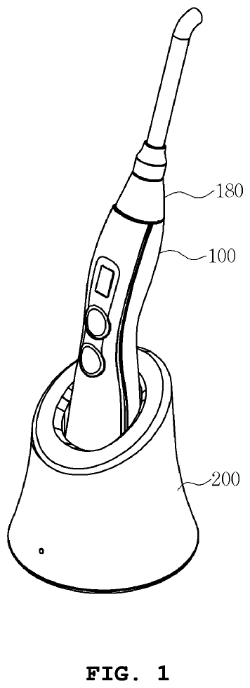

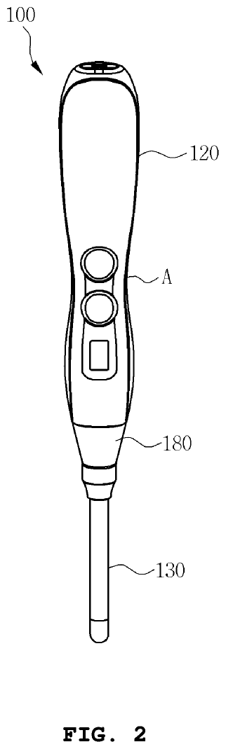

FIG. 1 is a perspective view showing a curing light with a heat dissipation structure according to the present invention, and FIGS. 2 and 3 are perspective views showing the curing light with a heat dissipation structure according to the present invention.

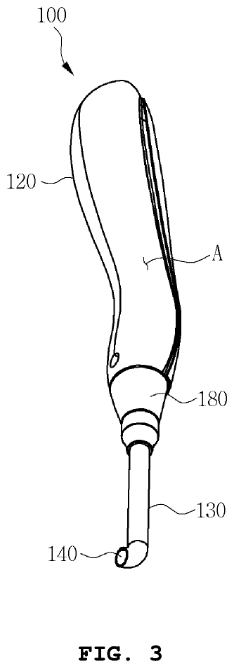

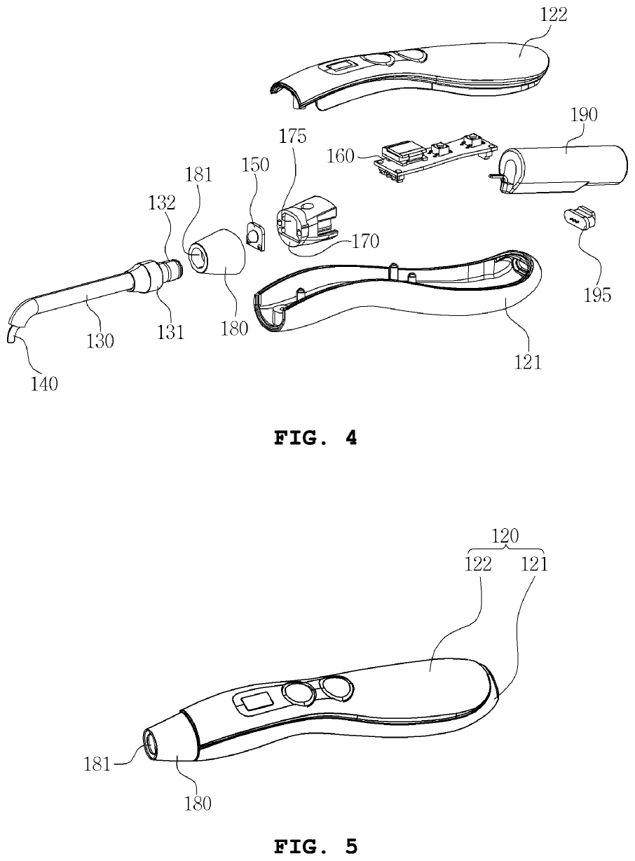

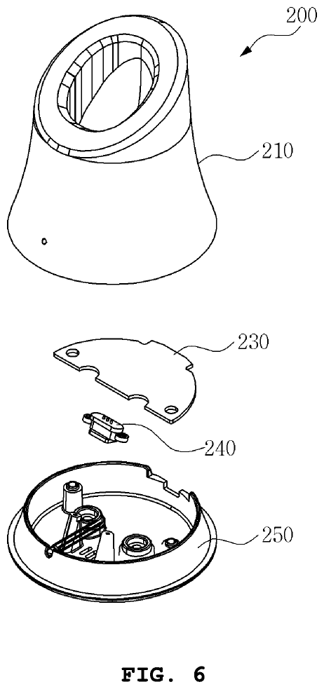

FIG. 4 is an exploded perspective view showing the curing light with a heat dissipation structure according to the present invention, FIG. 5 is a perspective view showing a coupled structure of a housing and heat sinks of the curing light with a heat dissipation structure according to the present invention, and FIG. 6 is an exploded perspective view showing a charging cradle of the curing light with a heat dissipation structure according to the present invention.

Hereinafter, an explanation on a curing light with a heat dissipation structure according to the present invention will be given in detail with reference to FIGS. 1 to 6.

Referring first to FIGS. 1 to 3, a curing light 100 with a heat dissipation structure according to the present invention is configured to be housed and charged in a charging cradle 200, and the curing light 100 has a handheld portion A curvedly formed on a housing 120 in such a way as to be easily held by a user.

Further, a head 130 is coupled to the end of the housing 120 and has an optical fiber 140 disposed therein.

The optical fiber 140 serves to irradiate the light emitted from a light source 150 as will be discussed later onto the user's teeth or gum. In specific, the optical fiber 140 provides the light emitted from the light source 150 located on the end of the housing 120.

The light source 150 is accommodated in the interior of the housing 120 and emits the light to be irradiated onto the user's teeth or gum. In this case, the light source 150 emits the light for curing a gum inflammation treatment material including a photosensitizer.

In more specific, the light source 150 is a high power LED (1.5 W/cm²) that emits the light with the wavelength (blue wavelength) of 450 nm to cure the gum inflammation treatment material including the photosensitizer.

Further, the optical fiber 140 may be provided to various forms to effectively emit the light to the user's teeth or gum. In specific, the optical fiber 140 may be formed of multiple optical fibers for uniformly irradiating light, or it may have optical fiber structure and arrangement corresponding to the shapes of the teeth and gum.

Next, an explanation of the internal configuration of the curing light 100 with a heat dissipation structure according to the present invention will be given in detail with reference to FIGS. 4 and 5.

The curing light 100 with a heat dissipation structure according to the present invention includes the housing 120, the head 130, the light source 150, a control board 160, first and second heat sinks 170 and 180, a battery 190, and a charging terminal 195.

The light source 150 emits the light for curing the gum inflammation treatment material including the photosensitizer, and the control board 160 controls the light source 150 according to the user's control command. Further, the housing 120 is configured to accommodate the light source 150, the control board 160, and the battery 190.

In this case, the first and second heat sinks 170 and 180 serve to dissipate the heat generated from the light source 150 and the control board 160.

In specific, the first heat sink 170 houses the light source 150 in a light source accommodation portion 175 to dissipate the heat generated from the light source 150.

Further, the first heat sink 170 is located inside the housing 120 and fixedly comes into close contact with the control board 160 to dissipate and transfer the heat generated from the control board 160 to the second heat sink 180.

Further, the second heat sink 180 is coupled to the first heat sink 170 to accommodate the light source 150, together with the first heat sink 170.

Further, the second heat sink 180 has a light transmission hole 181 formed thereon to transmit the light emitted from the light source 150 therethrough, and the outer surface of the second heat sink 180 is exposed to the outside.

According to the present invention, in more specific, the second heat sink 180 has the shape of a truncated cone, and the light transmission hole 181 has a light reflection surface treatment portion formed additionally on the peripheral surface thereof. The formation of the truncated cone of the second heat sink 180 enables the second heat sink 180 to be exposed to the outside, while allowing the curing light 100 to be easily held by the user, and the light reflection surface treatment portion serves to transmit the light to the head 130, while preventing the light from being lost inside the light transmission hole 181.

Further, the first and second heat sinks 170 and 180 are made by means of die casting and post-processing using aluminum.

Further, the head 130 is configured to accommodate the optical fiber 140 adapted to irradiate the light emitted from the light source 150 onto the outside, and a head fixing part 131 is provided to fix the head 130 thereto and thus coupled to the second heat sink 180 in such a way as to additionally dissipate the heat received from the second heat sink 180 to the outside.

In the same manner as the first and second heat sinks 170 and 180, the head fixing part 131 is made by means of die casting and post-processing using aluminum.

In this case, a coupling portion 132 is formed on the head fixing part 131 in such a way as to be fastened to a fastening portion formed on the inner peripheral end of the light transmission hole 181.

Further, the housing 120 is configured to have the first and second housings 121 and 122 coupled to each other, so that the light source 150, the control board 160, the battery 190, the charging terminal 195, and the first heat sink 170 are accommodated in the housing 120, and the second heat sink 180 is exposed to the outside from one side of the housing 120.

Moreover, the first and second housings 121 and 122 are made of polycarbonate (PC), and after they accommodate the light source 150, the control board 160, the battery 190, the charging terminal 195, and the first heat sink 170, they are firmly coupled to each other by means of ultrasonic welding.

Referring to FIG. 6, further, the charging cradle 200 of the curing light 100 with the heat dissipation structure according to the present invention includes a holder housing 210, a charging board 230, a charging connector 240, and a base housing 250.

The holder housing 210 fixedly mounts the curing light 100 and is coupled to the base housing 250.

The charging board 230 and the charging connector 240 are accommodated in the space formed by coupling the holder housing 210 and the base housing 250, and in this case, they are firmly fixed to the base housing 250 by means of fixing members such as screws.

Further, the holder housing 210 and the base housing 250 are made of polycarbonate (PC) and firmly coupled to each other by means of ultrasonic welding.

Under the above-mentioned configuration, the charging cradle 200 mounts the curing light 100 according to the present invention thereon and charges it.

As described above, the curing light with the heat dissipation structure according to the present invention can effectively dissipate the heat emitted from the light source and the heat generated from the control board to the outside through the heat dissipation structure in which the heat sinks are coupled to each other to accommodate the light source therein.

Further, the curing light with the heat dissipation structure according to the present invention can allow one side heat sink to be exposed to the outside, thereby having advantages such as conveniences of use and easy heat dissipation.

As mentioned above, the preferred embodiment of the present invention has been disclosed in the specification and drawings. Therefore, persons skilled in the relevant art can appreciate that many modifications and variations are possible in light of the above teachings. Therefore, it is manifestly intended that this invention be limited only by the claims and the equivalents thereof.

1. A curing light with a heat dissipation structure, comprising:
a light source for emitting light for curing a gum inflammation treatment material including a photosensitizer;
a control board for controlling the light source;
a housing for accommodating the light source and the control board; and
heat sinks for dissipating the heat generated from the light source,
wherein the heat sinks comprise: a first heat sink adapted to accommodate the light source and dissipate the heat generated from the light source; and a second heat sink coupled to the first heat sink in such a way as to accommodate the light source, together with the first heat sink, and having a light transmission hole through which the light emitted from the light source is transmitted.

2. The curing light according to claim 1, wherein the first heat sink is located inside the housing and fixedly comes into close contact with the control board to dissipate and transfer the heat generated from the control board to the second heat sink.

3. The curing light according to claim 2, wherein the outer surface of the second heat sink is exposed to the outside from the housing in which the light source and the control board are accommodated.

4. The curing light according to claim 3, wherein the second heat sink has the shape of a truncated cone, and the light transmission hole has a light reflection surface treatment portion formed on the peripheral surface thereof.

5. The curing light according to claim 4, further comprising:
a head for accommodating an optical fiber adapted to irradiate the light emitted from the light source onto the outside; and
a head fixing part for fixing the head thereto and coupled to the second heat sink in such a way as to dissipate the heat received from the second heat sink to the outside.

6. The curing light according to claim 5, wherein the second heat sink has a fastening portion formed on one side of the light transmission hole in such a way as to be fastened to a coupling portion formed on the head fixing part.

7. The curing light according to claim 4, wherein the first heat sink and the second heat sink are made by means of die casting and post-processing using aluminum.

8. The curing light according to claim 4, wherein the housing is configured to have first and second housings coupled to each other so that the light source, the control board, and the first heat sink are accommodated in the housing and the second heat sink is exposed to the outside from one side of the housing.
